(19) United States
(12) Patent Application Publication
(10) Pub. No.: US 2025/0242097 A1
(43) Pub. Date: Jul. 31, 2025

(54) SYSTEM AND METHOD FOR IMPROVING OXYGEN LEVEL IN THE BLOOD OF A HUMAN OR AN ANIMAL

(71) Applicants: KALYANARAMAN ANANTHANARAYANAN, TAMIL NADU (IN); DHEERAJ REDDY PYRA, TAMIL NADU (IN); GOKULAKRISHNAN MOHAN

(72) Inventors: KALYANARAMAN ANANTHANARAYANAN, TAMIL NADU (IN); DHEERAJ REDDY PYRA, TAMIL NADU (IN); GOKULAKRISHNAN MOHAN

(21) Appl. No.: 18/583,914

(22) PCT Filed: Aug. 25, 2022
(86) PCT No.: PCT/IB2022/057977

§ 371 Date: Feb. 22, 2024

(30) Foreign Application Priority Data
Aug. 25, 2021 (IN) 202141028729

(51) Int. Cl. A61M 1/16

(52) U.S. Cl. CPC A61M 1/1698; A61M 1/1678; A61M 2202/0429

(57) ABSTRACT

The present invention discloses an apparatus and a method to safely deliver oxygen into an environment of interest, such as blood plasma. The apparatus comprises an external oxygen source, a suitably insulated and reinforced tubular member, a diffuser head, an agitator, a vibrator and a collapsible cage member. The method employs deployment of a diffuser head which generates and releases ultrafine bubbles of gaseous oxygen or microdroplets of liquid oxygen, into a vascular chamber of interest which is the pulmonary artery, to improve oxygen levels in the blood contained in the vascular chamber.

### FIELD OF THE INVENTION

The present invention relates to medicine. Particularly, the invention discloses an apparatus, and a method for delivering an effective amount of oxygen to prevent systemic hypoxia and death in patients suffering from cardio-respiratory illnesses in the short and medium term, and to improve the quality of life in patients with severe cardio-respiratory disease in the long term. The apparatus and method can also be used to treat localised hypoxia in individual organ systems.

### BACKGROUND OF THE INVENTION

All animals including humans are dependent on oxygen for survival to the extent that humans cannot survive more than 2-3 minutes without oxygen. Oxygen is indispensable to maintain cellular structure and homeostasis. The physiological reserves of dissolved oxygen in the blood last only less than 5 minutes after which cardiac arrest and irreversible brain and other vital organ damage ensues.

At rest, an average human being requires about 250 ml of oxygen gas per minute for his basic metabolic needs. Any activity results in increased oxygen requirements. Engaging in vigorous physical activities like professional competitive sports may result in as much as 2500-4000 ml of oxygen getting 20 consumed per minute. Nonathletic human adults need about 250 mL/min of oxygen while at rest and about 500-1000 mL/min while engaging in day today physical activities. In a 24-hour period, the total oxygen consumed by an average, active adult is about 840 grams or 750 litres of oxygen gas, at normal temperature and pressure (S.T.P. i.e., 760 mm of Hg and 21° C.). This oxygen is absorbed from the 25 atmospheric air through the lungs, dissolves in the plasma, enters the red blood cells (“erythrocytes”) in the blood, binds to the haemoglobin (which is present inside the erythrocytes) and forms Oxyhaemoglobin. At any point of time, human body contains about 1100 ml of oxygen gas in the blood, of which about 98% is bound as oxyhaemoglobin inside the erythrocytes and the remaining 2% as dissolved oxygen in the plasma. At any point in time, the oxygen in the erythrocytes and the plasma are in a state of dynamic equilibrium, i.e., if the dissolved oxygen level decreases in the plasma, erythrocytes will release oxygen into the plasma and vice versa.

At tissue level, as oxygen from the plasma enters the cells, oxyhaemoglobin releases oxygen to the plasma to maintain the equilibrium and to continue further cellular oxygen delivery. Oxygen binding to haemoglobin is crucial to deliver physiologically significant amount of oxygen to the tissues as oxygen solubility in plasma is low and hence it cannot match the body requirements.

The functional unit of lungs is alveolus. Atmospheric air, with 21% oxygen, enters the alveoli during inspiration. Inside the alveoli, the oxygen concentration is about 13% (pO2 of 100 mm of Hg) and this comes into contact with the blood inside the pulmonary capillaries at the alveolar capillary junction. Alveolar capillary membrane acts as the interface for the gas exchange. Venous blood which is high in carbon dioxide reaches the pulmonary capillaries where it releases the CO2 into the alveolus and oxygen diffuses into the pulmonary capillaries from the alveolus to achieve a pO2 of about 98 mm of Hg in the plasma. Once oxygen dissolves in the plasma, it is immediately shifted into the erythrocytes where it binds to the haemoglobin. This action allows more oxygen to enter into the plasma, thereby allowing all the haemoglobin in the venous blood to get oxygenated in a single trip through the pulmonary circulation.

Acute shortage of oxygen in the human or animal body happens during various disease conditions like heart disease, lung disease, unconsciousness, anaesthesia, poisoning, epilepsy, pulmonary embolism etc., In these situations, oxygen levels in the blood are temporarily maintained by oxygen supplementation via face masks or tracheal tubes, with or without mechanical support (ventilator). These oxygen supplementation devices increase the oxygen concentration in the alveoli by replacing the alveolar nitrogen, and hence increase the pO2 in the alveolus up to about 500 mm of Hg or higher, thereby allows more oxygen to diffuse into the blood by displacing the dissolved nitrogen. In extreme situations, where the denitrogenating strategy is inadequate, the blood is taken out of the patient's body, pumped through an ‘Oxygenator’ device and pumped back into the patient's body.

This system is called Extracorporeal Membrane Oxygenation (ECMO) and can sustain oxygenation for days to weeks. But this has its own share of negatives like prohibitively high cost, prolonged ICU stay for weeks to months, blood loss, infection, end organ failure, loss of productive life etc. Patients who have end stage lung disease like interstitial lung disease, COPD, lung cancers requiring lung resections etc, will need lung transplant/heart-lung transplant, which has its share of negatives like prohibitively high cost, massive blood loss, high risk of surgical failure, need for lifelong immunosuppressants, extremely high demand for suitable donor organs etc.

Novel methods such as injectable oxygen gas in microbubbles (microbubbles are defined as bubbles having a diameter typically less than 1 mm, but predominantly around 10-500 micrometer), housed in an emulsion of lipids, surfactants and carrier liquids, as disclosed in US Patent US20120156300, have a limited but significant role in emergency resuscitation. They can sustain life only for a short time, typically about 30 minutes, and hence are not suitable for providing oxygen over hours to days or more. These emulsions typically contain about 50 ml of oxygen gas per dL and the other 50 ml will be lipids, surfactants and carrier molecules. Since the patient generally needs about 250 ml of oxygen gas at rest, to supply even 30% of that through this technique will mean that the emulsion will have to be infused at the rate of 150 ml per minute. This will result in acute intravascular volume expansion at the rate of 75 ml per minute (of lipids, surfactants and the carrier), which means more than 4500 ml of lipids, surfactants and the carrier liquid will accumulate in the bloodstream within an hour. This will put undue stress on the heart, liver and the kidneys and will result in a life-threatening situation.

More recently, in US patent publication US 2020/0261495 A1 the inventors have disclosed a method of oxygenating the blood using liquid oxygen which has been encapsulated in nanobubbles (nanobubbles are defined as bubbles having a diameter typically less than 1 micron) with lipids, surfactants and carrier medium. This technique also has too many additives like alcohol, surfactants etc, to manufacture and store the nanobubbles, which upon continuous infusion at physiological doses, will lead to unacceptable levels of these constituents endangering the life of the patient. Moreover, infusion of physiologically significant doses of oxygen (at least 100 ml of oxygen gas per minute) will require the infusion of too much of carrier medium which will result in intravascular volume overload and cardiac failure, particularly in the sick and elderly.

To overcome the problems of injecting carrier medium and the use of additives and emulsifiers, more recently, in US patent application US 2022/0080106 A1, the inventors have disclosed a method of intravascular oxygenation by directly generating oxygen microbubbles in the blood plasma of a human or an animal, typically housed inside the veins of the body, typically veins like jugular, femoral or the superior or inferior vena cava. This technique besides addressing the venous return only from one portion of the body (and not the complete quantity of venous blood, thereby reducing its therapeutic effectiveness), always has a risk of air bubbles entering systemic veins like cerebral, hepatic or intestinal veins creating ischaemic damage.

The problem of refractory hypoxemia in a human or animal is generally treated with oxygen supplementation using ventilator support or ECMO as described earlier. There are certain recent attempts to administer extracorporeally synthesised ultrafine oxygen bubbles (microbubbles and nanobubbles) into the human blood to improve the oxygenation. These extracorporeally synthesised oxygen microbubbles are stable in solution because they are bound by surfactants and stabilised in a carrier medium. They have been proven to release oxygen to the blood to maintain oxygenation for short periods (typically less than 30 minutes). Microbubbles by definition can mean any bubble of less than 1 millimeter diameter, but in the present context, it generally refers to bubbles with a diameter of 50-100 micrometers. Nanobubbles are defined as having a diameter less than 1 micrometer.

The ultrafine bubbles which we use for delivering oxygen to blood plasma, are typically less than 10 micrometers in diameter. This is important to prevent the occlusion of pulmonary vasculature, whose capillaries have a diameter of about 10 micrometers. The main disadvantage of injecting of such externally synthesised oxygen microbubbles is that the same surfactant molecules and the carrier medium which stabilise the oxygen microbubbles are the handicap for using this technique for prolonged periods as they result in fluid overload, systemic toxicity due to surfactant molecules and carrier molecules etc.

Hence, there is a need to provide an improved method for delivering oxygen to patients, tissues or organs, using oxygen gas or liquid oxygen with little or no additives, both in hospital and home care settings, for short-, medium-and 15 long-term oxygen dependent patients, without sacrificing their productive lifestyle.

### OBJECT OF THE INVENTION

The primary object of the invention is to improve oxygen levels in the blood of a human or an animal by local generation of ultrafine oxygen bubbles (micro and nano bubbles) directly in the bloodstream (instead of injecting micro/nanobubbles generated outside the body) using direct administration of oxygen gas or liquid oxygen into blood.

Yet another aspect of the invention pertains to an apparatus to deliver oxygen gas or liquid oxygen with little or no additives to patients, tissues or organs.

Yet another object of the invention pertains to delivering an effective amount of oxygen to prevent systemic hypoxia and death in patients suffering from cardio-respiratory illnesses in the short and medium term, and to improve the quality of life in patients with severe cardio-respiratory disease in the long term.

Yet another object of the invention pertains to treat localised hypoxia in individual organ systems.

Yet another object of the invention pertains to transmit liquid oxygen from an external reservoir and release microdroplets of liquid oxygen into the pulmonary artery, right atrium or right ventricle.

These and other objects and advantages of the present invention will become readily apparent from the following detailed description taken in conjunction with the accompanying drawings.

### SUMMARY OF THE INVENTION

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

According to an embodiment of the present invention is an apparatus to deliver oxygen inside a cardiovascular chamber, comprising a tubular catheter member having a proximal end (1), a body (2), and a distal end (3) and at least one inner lumen (7) extending from the proximal end (1) till the distal end (3). The proximal end (1) of the tubular catheter member lies outside the body of a human or animal and is connected to an oxygen source (9), allowing oxygen gas to flow through its lumen (7) and the distal end (3) of the tubular catheter member is disposed in a cardiovascular chamber (20) and comprises of a diffuser head (4), an agitator (6) and a vibrator (5) to deliver ultrafine oxygen gas bubbles. And in the case of the delivery of microdroplets of liquid oxygen to cardiovascular chambers (20), the distal end is provided with a collapsible cage (15). which allows compression, enabling its insertion into a cardiovascular chamber (20) through a small skin incision and upon favourable disposition in a compartment of interest, it regains its shape. The said collapsible cage encloses the diffuser head circumferentially and ensures there is no direct contact of liquid oxygen with the walls of the cardiac chambers.

According to the embodiment of the present invention, the said cardiovascular chamber (20) is a pulmonary artery or right ventricle or right atrium, thereby allowing adiabatic compression of oxygen microbubbles by utilising the blood velocity in the chamber.

According to the embodiment of the present invention, the oxygen source (9) is an oxygen cylinder or a liquid oxygen reservoir or any such oxygen generator or storing device coupled to a pump which is capable of delivering pressurised oxygen or liquid oxygen into the inner lumen of the tubular catheter member.

According to the embodiment of the present invention, the diffuser head (4) is made of a porous material made of a carbon-based material like carbon ceramic or graphite or other porous materials like ceramic, pumice stones or synthetic porous materials having an average pore diameter of less than 1 micrometre. Furthermore, the diffuser head (4) inner surface is connected with the inner lumen of the tubular catheter member, whereby the oxygen gas from the inner lumen diffuses though the pores of the diffuser head (4) to generate and release oxygen microbubbles into the cardiovascular chamber (20) in a direction perpendicular to the direction of blood flow, enabling adiabatic compression of the oxygen microbubbles into ultrafine bubbles (diameter less than 10 micrometer) or nanobubbles (diameter less than 1 micrometer).

According to the embodiment of the present invention, the agitator (6) is an electrically powered high frequency transducer housed near the distal end (3) of the tubular catheter member to enable agitation of the blood inside the cardiovascular chamber (20), thereby forming and breaking microbubbles, increasing surface area for absorption into red blood cells and preventing coalescence.

According to the embodiment of the present invention, wherein the vibrator (5) is an electrically driven component which vibrates the diffuser head (4) in such a way to mechanically disrupt and prematurely release the oxygen microbubbles from the surface of the diffuser head when they are still in the hemispherical phase, to keep the size of microbubbles small enough to prevent coalescence.

According to the embodiment of the present invention, for the delivery of microdroplets of liquid oxygen inside a cardiovascular chamber, the insulated tubular catheter member (14) is made of marine grade stainless steel or similar material which can withstand cryogenic temperatures and the insulation is done by means of vacuum insulation technology, Aerogel or a combination of similar techniques.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

FIG. 1 illustrates a preferred embodiment of an intravascular oxygenation device according to an aspect of the present disclosure.

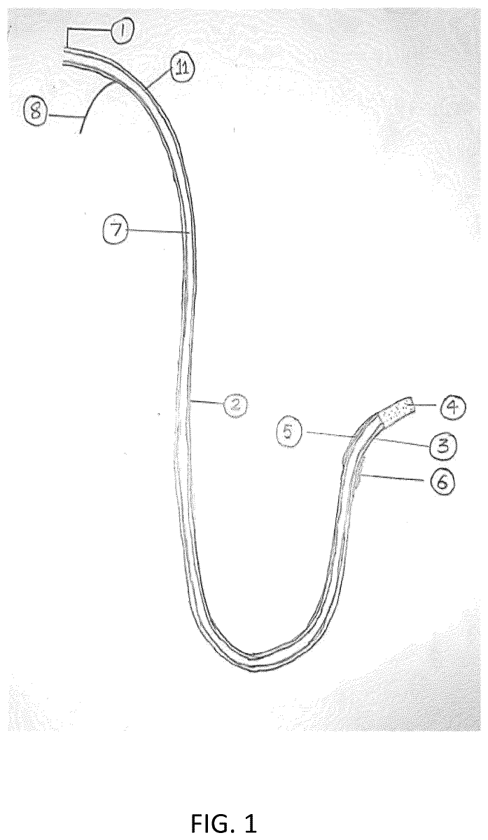

FIG. 2 illustrates the preferred embodiment in conjunction with an oxygen source, which is an oxygen cylinder.

FIG. 3 illustrates the transverse cut section at the level of the body of the tubular member, showing its components.

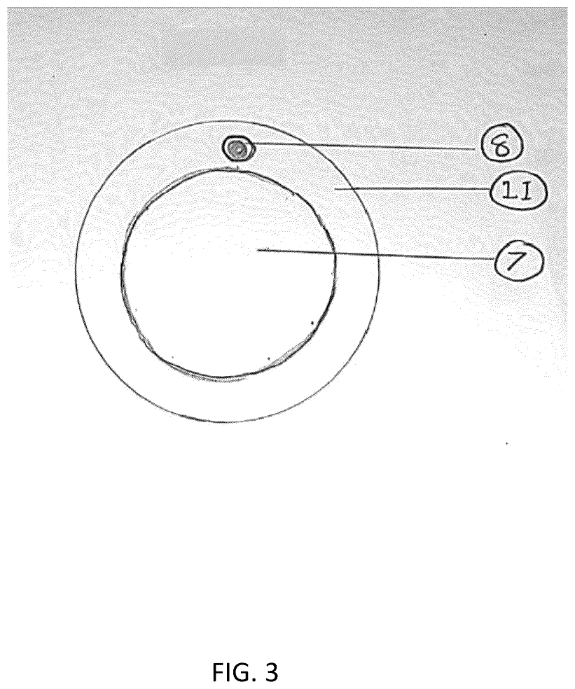

FIG. 4 illustrates a longitudinal section of the distal end of the device, revealing the longitudinally cut view of the diffuser head and its relation with the inner lumen, agitator and the vibrator apparatus.

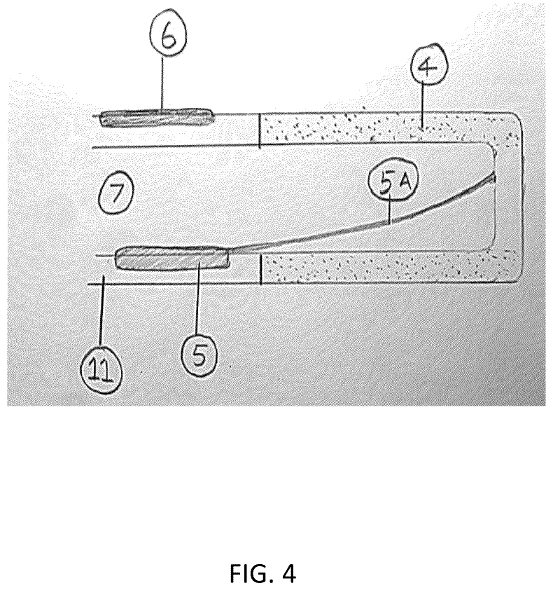

FIG. 5 is an illustration of the preferred embodiment of the disclosed device with its distal end housing the diffuser head favourably disposed in the pulmonary artery of a human subject.

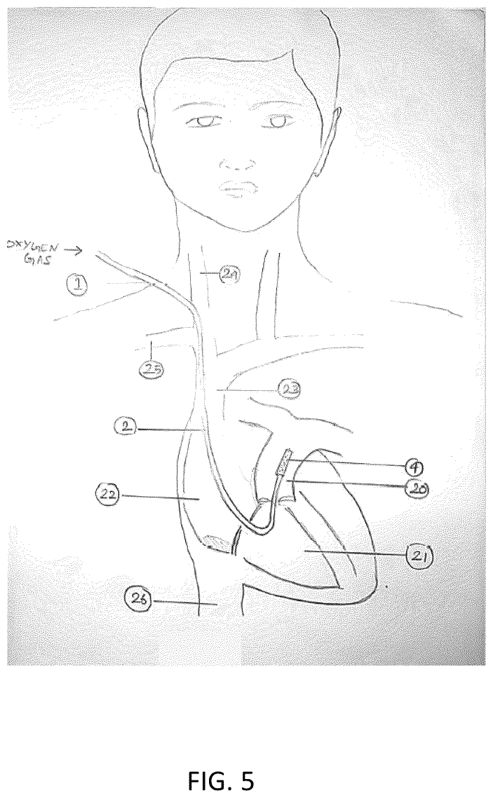

FIG. 6 is an illustration depicting the distal end of the disclosed device housed inside the pulmonary artery of a human, and the effect of the agitator and the vibrator apparatus to the diffuser head and the surrounding blood.

FIG. 7 is an illustration depicting the adiabatic compression of the microbubbles to ultrafine bubbles/nanobubbles when exposed to the pulmonary artery blood flow.

FIG. 8 is an illustration depicting the various stages of microbubble release from the diffuser head, showing gradual enlargement from hemispherical to spherical microbubbles while being released into the pulmonary artery.

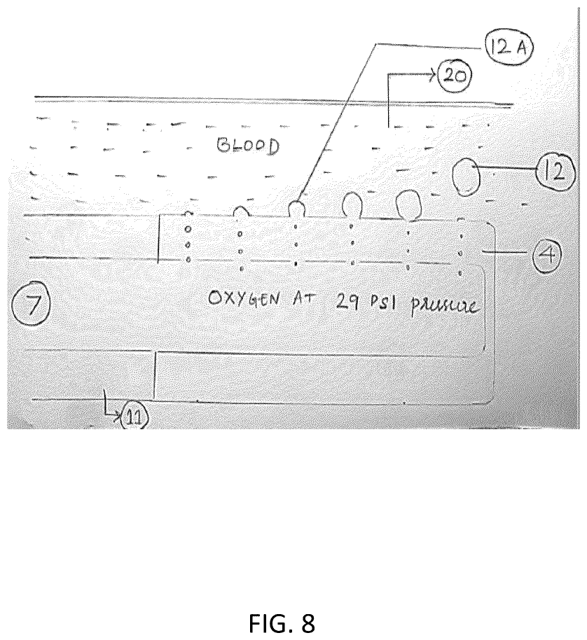

FIG. 9 is an illustration of an alternate embodiment of the disclosed device wherein, the device is modified to release microdroplets of liquid oxygen into the blood housed inside a cardiovascular chamber of a human or animal.

FIG. 10 is an illustration of the alternate embodiment of the disclosed device with its distal end housing the diffuser head and a collapsible cage favourably disposed in the pulmonary artery of a human subject.

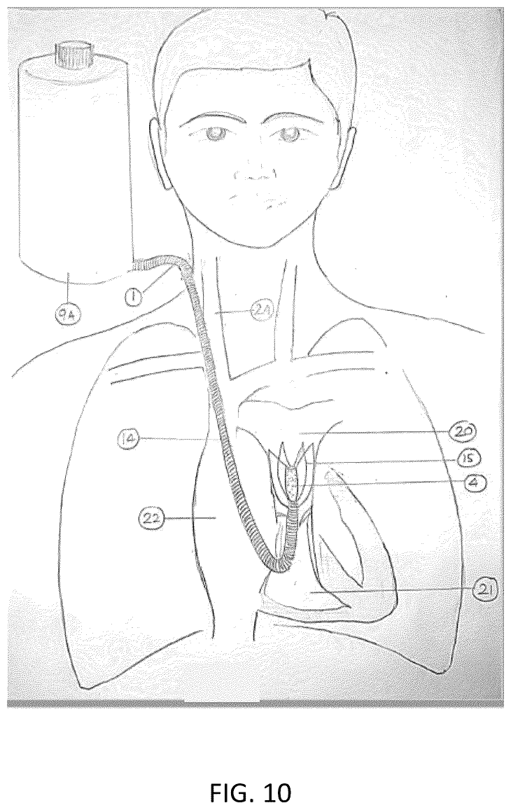

FIG. 11 is an illustration of the alternate embodiment of the disclosed device with its distal end housing the diffuser head and a collapsible cage favourably disposed inside the right atrium of a human subject.

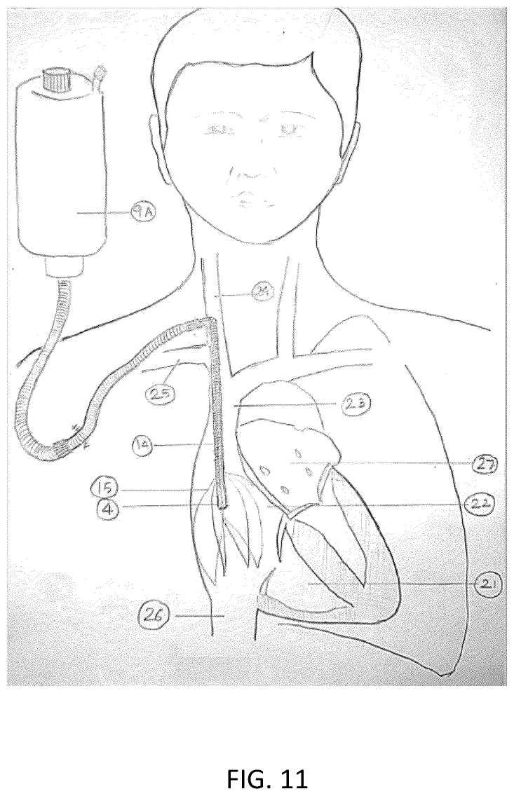

Although the specific features of the present invention are shown in some drawings and not in others. This is done for convenience only as each feature may be combined with any or all of the other features in accordance with the present invention.

### DETAILED DESCRIPTION OF THE INVENTION AND THE PREFERRED EMBODIMENTS

In the following detailed description, a reference is made to the accompanying drawings that form a part hereof, and in which the specific embodiments that may be practised are shown by way of illustration. These embodiments are described in sufficient detail to enable those skilled in the art to practice the embodiments and it is to be understood that other changes may be made without departing from the scope of the embodiments. The following detailed description is therefore not to be taken in a limiting sense. The various embodiments of the present invention constitute is an apparatus to deliver oxygen inside a cardiovascular chamber.

According to the challenges in generating bubbles intravascularly, as discussed in the background are mainly the size and the site. Microbubbles of size greater than 100 microns have a tendency to rise up in water (or blood) and also coalesce and form bigger bubbles which can occlude larger branches of pulmonary artery (20) leading to life threatening pulmonary embolism. This, coupled with the fact that the previous attempts had tried to generate oxygen microbubbles inside superior vena cava (23) and inferior vena cava (26), can result in the oxygen bubbles coalescing and rising through the venous system to result in venous embolism of cerebral, ophthalmic, hepatic or intestinal veins, resulting in significant potential morbidity.

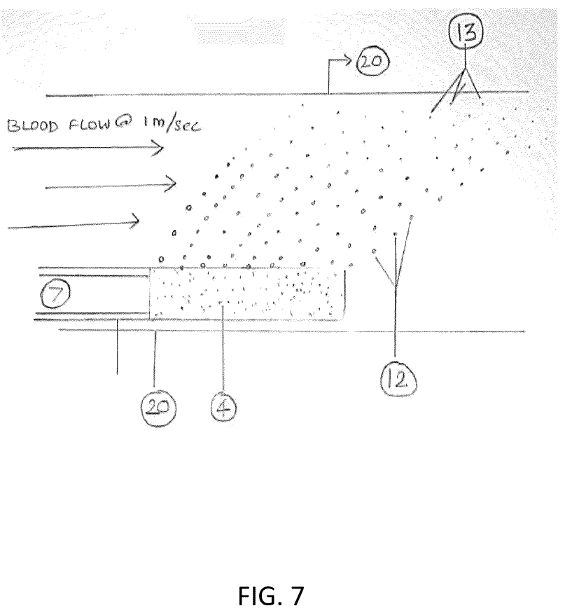

As illustrated in FIG. 7, the ultrafine oxygen bubbles (13) is directly generated in the blood of the human or animal rather than trying to inject the same from outside. the present embodiment provides for ultrafine bubbles (13) inside the pulmonary artery, which ensures that even in the unlikely event of coalescing of oxygen microbubbles (12), it will not result in cerebral, ophthalmic, hepatic or intestinal venous embolism.

The present embodiment provides for a mechanism for creating ultrafine bubbles (13) through dissolved air floatation, sonication, mechanical vibration, flow focusing and fluidic oscillation. Furthermore, the blood flowing through the right ventricle (21) of the heart and the pulmonary artery (20) have a velocity of about 1 meter/sec, providing the ideal environment to use this principle of ‘adiabatic compression’ inside the body of a human or animal.

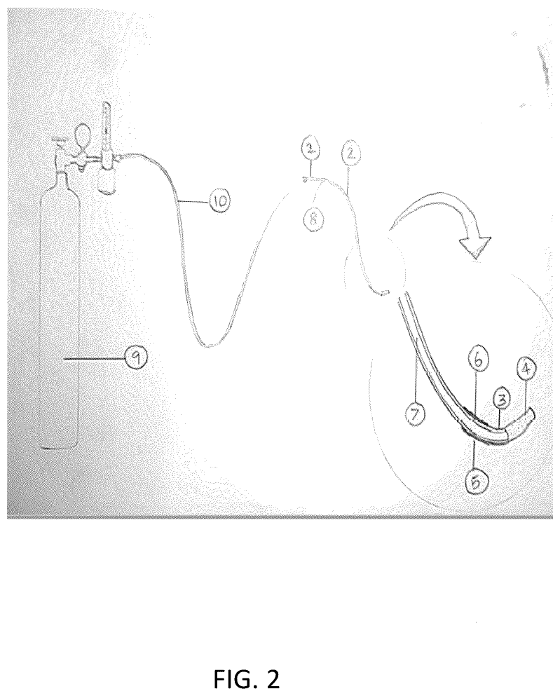

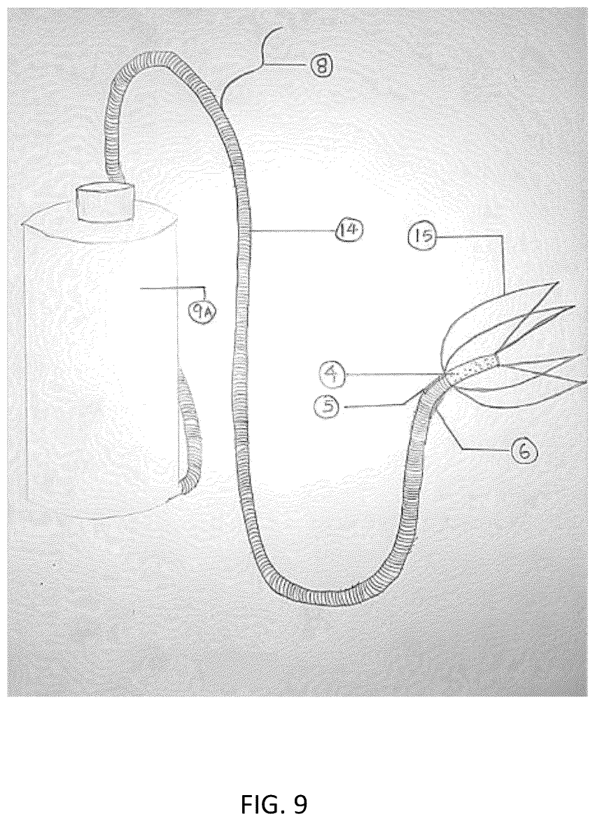

According to an embodiment of the present invention is an apparatus to deliver oxygen inside a cardiovascular chamber, comprising a tubular catheter member having a proximal end (1), a body (2), and a distal end (3) and at least one inner lumen (7) extending from the proximal end (1) till the distal end (3). The proximal end (1) of the tubular catheter member lies outside the body of a human or animal and is connected to an oxygen source (9), allowing oxygen gas to flow through its lumen (7) and the distal end (3) of the tubular catheter member is disposed in a cardiovascular chamber (20) and comprises of a diffuser head (4), an agitator (6) and a vibrator (5) to deliver ultrafine oxygen gas bubbles as illustrated in FIG. 2. And in the case of the delivery of microdroplets of liquid oxygen to cardiovascular chambers (20), the distal end is provided with a collapsible cage (15), as illustrated in FIG. 9, which allows compression, enabling its insertion into a cardiovascular chamber (20) through a small skin incision and upon favourable disposition in a compartment of interest, it regains its shape. The said collapsible cage encloses the diffuser head circumferentially and ensures there is no direct contact of liquid oxygen with the walls of the cardiac chambers.

The present embodiment provides for a carbon ceramic diffuser heads (4). Carbon ceramic looks and feels like a smooth stone, has an average pore size of less than 1 micrometer, and allows the generation of microbubbles of about 50 microns diameter at much lower inlet gas pressure (about 29 psi or 2 bars, as opposed to 5-6 bars for other diffuser systems). The oxygen gas is fed into the diffuser head at 29 psi pressure or less and exits from the whole surface of the diffuser head by osmosis. This diffuser head (4) is coupled with mechanisms for microbubble generation with ultrasound agitation (6), mechanical vibration (5), fluidic oscillation etc.

According to an embodiment, as illustrated in FIG. 2, the disclosed intravascular oxygenation method for generates and delivers ultrafine oxygen bubbles (13)/oxygen microbubbles (12) directly into a patient's vasculature through a catheter system. The catheter system has a tubular member with a proximal end (1), a body (2), a distal end (3) and at least one internal lumen (7) which traverses from the proximal end (1) to the distal end (2). The catheter can be inserted like a conventional Pulmonary Artery Catheter (Swan Ganz catheter), whereby, the catheter is inserted through a jugular (24), subclavian (25) or femoral vein, in such a way that its distal end (3) is disposed of favourably in the main pulmonary artery (20). The position of the distal end (3) can be confirmed by arterial pressure waveforms, echo cardiogram, roentgenograms or fluoroscopy.

According to the embodiment, the proximal end (1) of the catheter is connected to an oxygen source (9) which supplies pressurised Oxygen, preferably around 29 psi (2 bars), but can be much higher if the situation demands. The distal end (3) of the catheter houses a diffuser head (4), which is made of a suitable durable carbon-based material like carbon ceramic, ceramic, graphite or other similar porous material. The diffuser head (4) has an inner lumen (7) which receives the oxygen gas from the oxygen source and the outer surface of the diffuser head lies in contact with the blood inside the pulmonary artery. The average size of the pores in the porous diffuser head is less than 1 micron.

The oxygen gas which reaches the inner lumen (7) of the diffuser head, diffuses to the outer surface of the diffuser head, where it comes into contact with the blood. Upon coming to contact with the blood the oxygen gas initially forms a hemispherical bubble (12A) which grows into spherical bubble (12) a size of 10-50 microns before getting detached from the diffuser head. Upon releasing from the outer surface of the diffuser head, the microbubbles (12) are exposed to the blood flowing through the pulmonary artery (20) in a perpendicular direction (FIG. 7). Inside the pulmonary artery, blood flows with a peak velocity of 100 cm/sec during systole and about 30 cm/sec during diastole. This blood flow, upon coming into contact with the freshly formed oxygen microbubbles, transforms the microbubbles (12) into ultrafine bubbles (13)/nanobubbles by a principle named ‘adiabatic compression’, through which, the diameter of the microbubbles decreases to less than 10 microns, thereby, making them small enough to pass through the pulmonary capillaries and light enough to ensure they don't rise up and coalesce to form larger bubbles.

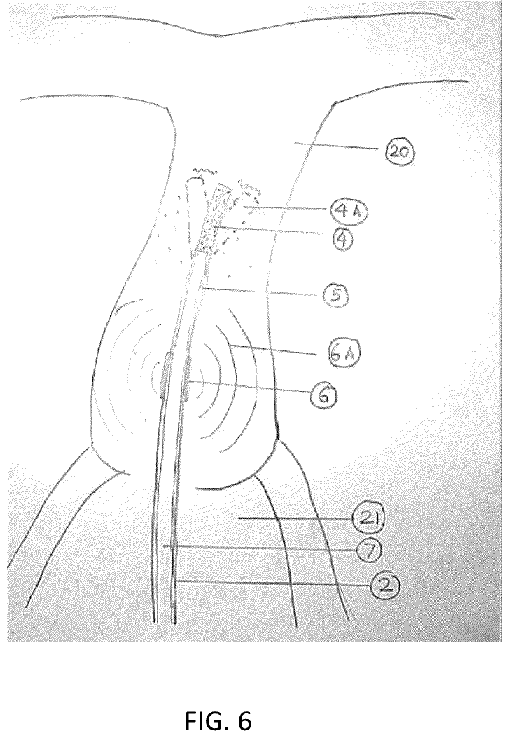

The present embodiment, apart from using a carbon-ceramic diffuser head (4) and the adiabatic compression, the device houses an agitator (6) and a vibrating system (5, 5A) to ensure the diameter of the oxygen bubbles stay below 10 microns. The agitator (6) and the vibrator (5) can be powered through an external power source through a cable wire (8) which is housed inside the wall of the tubular member and traverses from the proximal end (1) till near the distal end (3). The agitator (6) is an electrically powered high-frequency transducer which is designed to transmit the energy (6A) to the blood in the pulmonary artery, causing microbubbles to form and break continuously due to pressure variations as explained by ‘cavitation principle’, because of which there is an increase in oxygen absorption into red blood cells due to a high degree of increase in the surface area of contact between oxygen and blood plasma. This cavitation principle also creates turbulence and thereby increasing the distance between microbubbles, and thereby minimising the chance of the microbubbles rising up or coalescing to form larger bubbles. The vibrator (5) is an electrically powered transducer which is connected to the diffuser head through an anchor filament (5A) in such a way to enable vibration of the diffuser head (4A, FIG. 6) to ensure the microbubbles get released in the hemispheric phase (12A) itself, rather than allowing it to grow to its full size (12) before detachment from the diffuser head (4). In some embodiments, the agitator and vibrator may be integrated into a single part.

In another embodiment of the device, instead of oxygen gas, the device may be modified to transmit liquid oxygen from an external reservoir (9A) and release microdroplets of liquid oxygen into the pulmonary artery (20), right atrium (22) or right ventricle (21) through the diffuser head (4). In such an embodiment, the proximal end (1) of the tubular member will be having a one-way valve to prevent backflow, and the distal end (3) of the tubular member will house a collapsible cage (15) surrounding the diffuser head (4). The collapsible cage member (15) can be squeezed at the time of insertion of the device into the pulmonary artery (20)/right atrium, and upon placement into its desired location, it re-expands to its original shape. This compressibility allows the device to be introduced into the vascular compartment of a human or animal through a small skin incision, typically less than 1 cm. This collapsible cage (15) surrounds the diffuser head circumferentially for at least 1-2 cm and hence prevents any liquid oxygen droplet from coming into direct contact with the cardiac/vascular tissue, thereby preventing any tissue injury that may otherwise result from the cryogenic temperatures of the liquid oxygen (typically less than −183 degree Celsius). This cage member (15) can be made of Nitinol or other suitable material. Also, in this embodiment, the tubular member has an insulation jacket (14) running from its proximal end to the distal end, whereby the jacket prevents any tissue injury to any part of the body coming into direct contact with the tubular member, as the liquid oxygen will be carried inside the lumen (7) of the member maintaining a temperature of −183 degree Celsius or lower. This insulation jacket (14) may provide the necessary insulation using vacuum insulation technology or a technology like Aerogel or a combination thereof.

According to the embodiment each ml of liquid oxygen, when exposed to body temperature, gets converted to 866 ml of oxygen gas. Since a normal human being requires about 250 ml of oxygen at rest, even if the disclosed device has to provide 90% of the oxygen needs, only about 15 ml of liquid oxygen will need to be infused per hour which can be tolerated by most patients without any serious thermal side effects. The specific heat of liquid oxygen is 0.347 cal/g/C, heat of vaporisation is 51 cal/gram, and the specific heat of oxygen gas is 0.22 cal/g/C. Hence, heating 360 mL (@15 mL/hour) of liquid oxygen to 37 degree C. by the body, will need only about 42 Kcals, which is similar to the heat loss to the body caused by drinking 1300 ml of cold water (at 4 degree C.) over a period of 24 hours. Whatever the minimal drop in body temperature caused by the infusion of liquid oxygen can be overcome by the patient by drinking warm liquids and by wearing warm clothing and the liquid oxygen is cryogenic and highly reactive it will need to be stored and delivered using specialised alloys such a marine grade stainless steel or similar material. The reservoir (9A), tubing (14) and other parts of the apparatus which come in direct contact with the liquid oxygen will be made of such compatible material. Therefore, providing a suitable and seamless delivery mechanism without any adverse impact.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

1. An apparatus to deliver oxygen inside a cardiovascular chamber, comprising:
a tubular catheter member having a proximal end (1), a body (2), and a distal end (3) and at least one inner lumen (7) extending from the proximal end (1) till the distal end (3);
wherein the proximal end (1) of the tubular catheter member lies outside the body of a human or animal and is connected to an oxygen source (9), allowing oxygen gas to flow through its lumen (7);
and the distal end (3) of the tubular catheter member is disposed in a cardiovascular chamber (20, 21, or 22) and comprises of a diffuser head (4), an agitator (6) and a vibrator (5) to deliver ultrafine oxygen gas bubbles;
wherein for the delivery of microdroplets of liquid oxygen to cardio vascular chambers (20), the distal end is provided with a collapsible cage (15) and the tubular catheter member is suitably insulated (14)

2. The apparatus as claimed in claim 1, the said cardiovascular chamber is a pulmonary artery (20) or right ventricle (21) or right atrium (22), thereby allowing adiabatic compression of oxygen microbubbles by utilising the blood velocity in the chamber.

3. The apparatus as claimed in claim 1, wherein the diffuser head (4) is made of a porous material made of a carbon-based material like carbon ceramic or graphite or other porous materials like ceramic, pumice stones or synthetic porous materials having an average pore diameter of less than 1 micrometer.

4. The apparatus as claimed in claim 1, wherein the diffuser head (4)'s inner surface is connected with the inner lumen (7) of the tubular catheter member, whereby the oxygen gas from the inner lumen (7) diffuses though the pores of the diffuser head (4) to generate and release oxygen microbubbles into the cardiovascular chamber (20, 21 or 22) in a direction perpendicular to the direction of blood flow in the cardiovascular chamber, enabling adiabatic compression of the oxygen microbubbles into ultrafine bubbles (diameter less than 10 micrometer) or nanobubbles (diameter less than 1 micrometer).

5. The apparatus as claimed in claim 1 wherein the agitator (6) is an electrically powered high frequency transducer housed near the distal end (3) of the tubular catheter member to enable agitation of the blood inside the cardiovascular chamber (20, 21 or 22), thereby forming and breaking microbubbles, increasing surface area for absorption into red blood cells and preventing coalescence.

6. The apparatus as claimed in claim 1 wherein the vibrator (5) is an electrically driven component which vibrates the diffuser head (4) in such a way to mechanically disrupt and prematurely release the oxygen microbubbles from the surface of the diffuser head when they are still in the hemispherical phase (12A), to keep the size of microbubbles small enough to prevent coalescence.

7. The apparatus as claimed in claim 1 wherein the vibrator (5) is an electrically powered transducer which is connected to the diffuser head through an anchor filament (5A), to enable vibration of the diffuser head (4) to ensure the microbubbles get released in the hemispheric phase (12A).

8. The apparatus as claimed in claim 1, wherein the body (2) of the tubular catheter member is made of a suitably flexible and reinforced material and houses an inner lumen (7) to transmit oxygen from the proximal end (1) to the diffuser head (4).

9. The apparatus as claimed in claim 1 wherein the body of the tubular catheter member houses an insulated electric cable wire (8) extending along its wall, wherein the external end of the electric cable wire is connected to an external power source and the other end (internal end) of the electric cable wire is connected to the agitator (6) and vibrator (5).

10. The apparatus as claimed in claim 1 wherein the oxygen source (9) is an oxygen cylinder or a liquid oxygen reservoir (9A) or any such oxygen generator or storing device coupled to a pump which is capable of delivering pressurised oxygen gas or liquid oxygen into the inner lumen of the tubular catheter member.

11. The apparatus as claimed in claim 1, wherein for the delivery of microdroplets of liquid oxygen inside a cardiovascular chamber, the insulated tubular catheter member (14) is made of marine grade stainless steel or similar material which can withstand cryogenic temperatures (<−183 degree Celsius) and the insulation is done by means of vacuum insulation technology, Aerogel or a combination of similar techniques.

12. The apparatus as claimed in claim 1, wherein for the delivery of microdroplets of liquid oxygen inside a cardiovascular chamber the collapsible cage (15) is made of Nitinol or similar alloy which allows compression, enabling its insertion into a cardiovascular chamber (20) through a small skin incision and upon favourable disposition in a compartment of interest, it regains its shape.

13. The apparatus as claimed in claim 1, wherein the collapsible cage (15) 1encloses the diffuser head circumferentially for at least 1-2 centimeters and ensures there is no direct contact of liquid oxygen with the walls of the cardiac chambers.

14. A method for safely and reliably delivering oxygen into the blood plasma of a human or animal, comprising of directly generating ultrafine bubbles of oxygen gas or delivering microdroplets of liquid oxygen in the pulmonary artery, right atrium or the right ventricle of the human or animal by infusing oxygen gas or liquid oxygen through a diffuser head at a flow rate which allows the dissolution of oxygen in the plasma and its absorption into erythrocytes, wherein said human or animal is experiencing local or systemic hypoxia due to disease, accidents or poisoning, wherein oxygen gas or liquid oxygen is administered in an effective amount to increase the concentration of oxygen in the patient's blood, tissue or organ in need of oxygen, to physiologic levels, completely or partially bypassing the work of respiratory system and the lungs.
